(19) United States
(12) Patent Application Publication
(10) Pub. No.: US 2014/0107204 A1
(43) Pub. Date: Apr. 17, 2014

(54) METHOD OF ADMINISTERING ETHANOLAMINE 9-OCTADECANOIC ACID VIA INJECTION TO THE STOMACH OF A HUMAN

(71) Applicant: QOL Medical LLC, Vero Beach, FL (US)

(72) Inventors: Dayton T. Reardan, Shorewood, MN (US); Derick Cooper, Vero Beach, FL (US)

(73) Assignee: QOL Medical LLC, Vero Beach, FL (US)

(21) Appl. No.: 13/961,501

(22) Filed: Aug. 7, 2013

Related U.S. Application Data
(60) Provisional application No. 61/680,919, filed on Aug. 8, 2012.

(51) Int. Cl. A61K 31/201; A61K 31/133

(52) U.S. Cl. CPC A61K 31/201; A61K 31/133
USPC 514/560; 600/104

(57) ABSTRACT

The present invention provides for a method that includes safely administering via injection ethanolamine and 9-octadecanoic acid, a pharmaceutically acceptable thereof, or a pharmaceutical formulation including the same, to the stomach of a human, e.g., to prevent weight loss reversion, to induce weight loss, and/or control weight loss.

### CLAIM OF PRIORITY

This patent application claims the benefit of priority to U.S. Provisional Patent Application Ser. No. 61/680,919, entitled “METHOD OF ADMINISTERING ETHANOLAMINE TO THE GASTROJEJUNOSTOMY STOMA OF A HUMAN, FOLLOWING BARIATRIC SURGERY,” filed on Aug. 8, 2012, which is hereby incorporated by reference herein in its entirety.

### BACKGROUND

Obesity now affects 90 million Americans,¹ contributes to 300,000 deaths annually, and is believed to be the second most preventable cause of death in the United States.² When therapeutic changes in diet and lifestyle fail, many morbidly obese patients seek bariatric surgery, which is now performed more than 200,000 times annually.³ Gastric bypass is the most commonly performed bariatric procedure (institutional data from a survey to the American Society for Metabolic and Bariatric Surgery members; unpublished data), resulting in the initial loss of an average of 57-67% of the excess weight.⁴ ¹National Heart, Lung, and Blood Institute (BHBLI). Clinical guidelines on the identification, evaluation, and treatment of overweight and obesity in adults: the evidence report. Obes Res 1998;6(Suppl 2):51-2098.²Hill J O, Wyat H R, Reed G W, et al. Obesity in the environment, where do we go from here? Science 2003;299:853-5³Zhao Y, Encinosa W. Bariatric surgery utilization and outcomes in 1998 and 2004, statistical brief #23. Agency for Healthcare Research and Quality 2007.⁴Buchwald H, Avidor Y, Braunwald E, et al. Bariatric surgery: a systematic review and met-analysis. JAMA 2004;292:1724-37

Maintaining weight loss and the prevention of regaining weight after any bariatric procedure is a struggle for many patients. The exact prevalence of this problem in Roux-en-Y gastric bypass patients is difficult to determine due to incomplete long-term follow-up after surgery. However, practitioners have found this to be a common problem in their post-bariatric surgery patients, especially beyond 2 years after surgery. Often the weight gain results from some combination of the patient's dietary indiscretion, lack of exercise, or genetic profile. It can also be secondary to surgical complications such as staple line disruption, a gastrogastric fistula, pouch dilation, or dilation of the gastric pouch and the jejunum referred to as the gastrojejunal anastomosis (GJA). Surgical revision for bariatric surgery patients that have regained weight, whether from dietary factors or from mechanical complications such as a dilated GJA, has generally had a high risk/benefit ratio.⁵ Prevention of a dilated GJA with silastic rings also carries a risk of significant emesis, erosion, and migration of the rings.⁶ ⁵Martin M J, Mullenix P S, Steele S R, See C S, Cuadrado D G, Carter P L. A case-match analysis of failed prior bariatric procedures converted to resectional gastric bypass. Am J Surg 2004;187:666 -71; Holzwarth R, Huber D, Majkrzak A, Tareen B. Outcome of gastric bypass patients. Obes Surg 2002;12:261-4; MacLean L D, Rhode B M, Nohr C W. Late outcome of isolated gastric bypass. Ann Surg 2000;231:524-8; Yale C E. Gastric surgery for morbid obesity: complications and long-term weight control. Arch Surg 1989;124:941-6; and Schwartz R W, Strodel W E, Simpson W S, Griffen W O. Gastric bypass revision: lessons learned from 920 cases. Surgery 1988;104:806-12.⁶Salinas A, Santiago E, Yegüez J, Antor M, Salinas H. Silastic ring vertical gastric bypass: evolution of an open surgical technique, and review of 1,588 cases. Obes Surg 2005;15:1403-7; Fobi M A, Lee H. Silastic ring vertical banded gastric bypass for the treatment of obesity. J Natl Med Assoc 1994;86:125-8; Crampton N A, Izvomikov V, Stubbs R S. Silastic ring gastric bypass: results in 64 patients. Obes Surg 1997;7:489-94.

Endoscopic sclerotherapy of the GJA with sodium morrhuate has been described as a minimally invasive technique for narrowing the diameter of the GJA. The morrhuate technique and 3-6-month outcomes have been previously reported.⁷ ⁷Spaulding L. Treatment of dilated gastrojejunostomy with sclerotherapy. Obes Surg 2003;13:254-7.

### BRIEF DESCRIPTION OF THE FIGURES

FIG. 1 illustrates a gastrojejunostomy.

FIG. 2 illustrates a type of gastric bypass surgery referred to as a “Roux-en-Y gastric bypass” or “RYGB” surgery.

FIG. 3 illustrates a photo, showing the injection into the fundus stomach region of a pig with ethanolamine oleate, in various embodiments.

FIG. 4 illustrates a photo, showing the injection into the fundus stomach region of a pig with saline (control), in various embodiments.

FIG. 5 illustrates a photo, showing the injection into the cardia stomach region of a pig with ethanolamine oleate, in various embodiments.

FIG. 6 illustrates a photo, showing the injection into the fundus and cardia stomach region of a pig with saline (control), in various embodiments.

FIG. 7 illustrates a photo, showing the injection into the fundus and cardia stomach region of a pig with 5% ethanolamine oleate (close up), in various embodiments.

### SUMMARY

The present invention provides for a use and method for administering, via injection, ethanolamine and 9-octadecanoic acid, or a pharmaceutically acceptable thereof (e.g., ethanolamine oleate), as a clear sterile parenterally acceptable solution, to the stomach of a human. The present invention also provides these compositions for use in a method for treatment of the human or animal body by surgery or therapy, as well as for the specific use in certain methods of treatment.

The present invention also provides for a method that includes administering ethanolamine and 9-octadecanoic acid, or a pharmaceutically acceptable salt thereof (e.g., ethanolamine oleate), to the stoma of a human.

The present invention also provides for a method that includes administering ethanolamine and 9-octadecanoic acid, or a pharmaceutically acceptable thereof (e.g., ethanolamine oleate), to the banded stomach of a human created via a bariatric banding surgical procedure.

The present invention also provides for a method that includes administering ethanolamine and 9-octadecanoic acid, or a pharmaceutically acceptable thereof (e.g., ethanolamine oleate), to the stomach or stoma of a human created via other bariatric surgical procedures intended to induce malabsorption and/or lower thresh-holds of postprandial satiety to increase weight loss in morbidly obese patients.

The present invention also provides for a method that includes administering ethanolamine and 9-octadecanoic acid, or a pharmaceutically acceptable thereof (e.g., ethanolamine oleate), to a normal human stomach prior to or instead of bariatric surgery in severely or morbidly obese patients.

Collectively, any procedure involving the use of ethanolamine and 9-octadecanoic acid, or a pharmaceutically acceptable thereof (e.g., ethanolamine oleate), (i) prior to, (ii) instead of, (iii) during, and/or (iv) after any bariatric surgical procedure is intended to induce malabsorption and/or lower thresh-holds of postprandial satiety to increase weight loss in morbidly obese patients.

The present invention also provides for a method of decreasing the diameter of the GJA, the stoma, the pyloric sphincter or the banded fundus in bariatric surgeries in a bariatric surgical procedure. The method includes administering an amount of ethanolamine and ethanolamine and 9-octadecanoic acid, or a pharmaceutically acceptable thereof (e.g., ethanolamine oleate) to the stoma or stomach of a human. The amount of ethanolamine and 9-octadecanoic acid, or a pharmaceutically acceptable salt thereof (e.g., ethanolamine oleate), is effective in decreasing (or stabilizing) the diameter of the GJA, stoma or stomach in a human who has received a bariatric surgical procedure.

The present invention also provides for a method of stabilizing the weight of a human patient having previously experienced a bariatric surgical procedure. The method includes administering an amount of ethanolamine and 9-octadecanoic acid, or a pharmaceutically acceptable salt thereof (e.g., ethanolamine oleate), to the stoma or stomach of a human. The amount of ethanolamine and 9-octadecanoic acid, or a pharmaceutically acceptable salt thereof (e.g., ethanolamine oleate) is effective in stabilizing the weight of a human patient.

The present invention also provides for a method of increasing weight loss in a human patient having previously experienced a bariatric surgical procedure. The method includes administering an amount of ethanolamine and 9-octadecanoic acid, or a pharmaceutically acceptable salt thereof (e.g., ethanolamine oleate), to the stoma or stomach of a human. The amount of ethanolamine and 9-octadecanoic acid, or a pharmaceutically acceptable salt thereof (e.g., ethanolamine oleate), is effective in increasing the weight loss of a human patient.

The present invention also provides for a method of decreasing weight gain in a human patient having previously experienced a bariatric surgical procedure. The method includes administering an amount of ethanolamine and 9-octadecanoic acid, or a pharmaceutically acceptable thereof (e.g., ethanolamine oleate) to the stoma or stomach of a human. The amount of ethanolamine and 9-octadecanoic acid, or a pharmaceutically acceptable thereof (e.g., ethanolamine oleate) is effective in decreasing the weight gain of a human patient.

The present invention also provides for a method of decreasing recurrent weight gain in a human patient having previously experienced a bariatric surgical procedure. The method includes administering an amount of ethanolamine and 9-octadecanoic acid, or a pharmaceutically acceptable thereof (e.g., ethanolamine oleate) to the stoma or stomach of a human. The amount of ethanolamine and 9-octadecanoic acid, or a pharmaceutically acceptable thereof (e.g., ethanolamine oleate) is effective in decreasing the recurrent weight gain of a human patient.

The present invention also provides for a method of preventing recurrent weight gain in a human patient having previously experienced a bariatric surgical procedure. The method includes administering an amount of ethanolamine oleate to the stoma or stomach of a human. The amount of ethanolamine and 9-octadecanoic acid, or a pharmaceutically acceptable thereof (e.g., ethanolamine oleate) is effective in preventing the recurrent weight gain of a human patient.

The present invention also provides for a method of reducing stoma or stomach size and expansion in a human patient having previously experienced a bariatric surgical procedure. The method includes administering an amount of ethanolamine oleate to the stoma or stomach of a human. The amount of ethanolamine and 9-octadecanoic acid, or a pharmaceutically acceptable thereof (e.g., ethanolamine oleate) is effective in reducing the stoma or stomach size and expansion of a human patient.

The methods described herein are generally safe and effective for the desired indication of use.

The present invention also provides for a composition that includes ethanolamine and 9-octadecanoic acid, or a pharmaceutically acceptable thereof (e.g., ethanolamine oleate). The present invention also provides these compositions that include ethanolamine and 9-octadecanoic acid, or a pharmaceutically acceptable thereof (e.g., ethanolamine oleate) for use in a method for treatment of the human or animal body by surgery or therapy. The composition is configured for injection to the stomach of a human. The invention is also directed to the specific use of these compositions that include ethanolamine and 9-octadecanoic acid, or a pharmaceutically acceptable thereof (e.g., ethanolamine oleate) in a method of at least one of: decreasing the diameter of the GJA in a human patient; reducing stoma size in a human patient; reducing the gastric pouch size in a human patient; reducing stoma expansion in a human patient; hardening the stomach in a human patient; stabilizing the weight of a human patient; increasing weight loss in a human patient; decreasing weight gain in a human patient; decreasing recurrent weight gain in a human patient; preventing recurrent weight gain in a human patient; preventing diabetes in a human patient; decreasing the caloric intake in a human patient;

decreasing food intake in a human patient; decreasing a meal size consumed by a human patient; decreasing the number of meals consumed by a human patient; decreasing the appetite of a human patient; inhibiting the action of a hunger-stimulating hormone in a human patient; inhibiting the action of an appetite-regulating hormone in a human patient; inhibiting the action of ghrelin in a human patient; and lowering the level of ghrelin in a human patient.

### DETAILED DESCRIPTION

Reference will now be made in detail to the invention, certain embodiments of which are illustrated below. While the invention will be described in conjunction with the enumerated claims, it will be understood that the description is not intended to limit the subject matter recited in the claims. On the contrary, reference to the invention is intended to cover all alternatives, modifications, and equivalents, which may be included within the scope of the subject matter as defined by the claims.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The present invention generally relates to administering ethanolamine and 9-octadecanoic acid, or a pharmaceutically acceptable salt thereof (e.g., ethanolamine oleate) to the stoma or stomach of a human either (i) prior to, (ii) instead of, (iii) after, or (iv) during any bariatric procedure intended to induce weight loss via malabsorption, reduced caloric intake and/or reduced postprandial satiety thresh-holds. When describing such an invention, the following terms have the following meanings, unless otherwise indicated.

### DEFINITIONS

Unless stated otherwise, the following terms and phrases as used herein are intended to have the following meanings:

The term “stomach” refers to a muscular, hollow, dilated part of the digestion system which functions as an important organ of the digestive tract in humans. It is involved in the second phase of digestion, following mastication (chewing). The stomach lies between the esophagus and the duodenum (the first part of the small intestine). It is on the left upper part of the abdominal cavity. The top of the stomach lies against the diaphragm. Lying behind the stomach is the pancreas. The greater omentum hangs down from the greater curvature. Two sphincters keep the contents of the stomach contained. They are the esophageal sphincter (found in the cardiac region, not an anatomical sphincter) dividing the tract above, and the pyloric sphincter dividing the stomach from the small intestine. The stomach is divided into four sections, each of which has different cells and functions. The sections are: (1) cardia (where the contents of the esophagus empty into the stomach), (2) fundus (formed by the upper curvature of the organ), (3) body or corpus (the main, central region), and (4) pylorus (the lower section of the organ that facilitates emptying the contents into the small intestine).

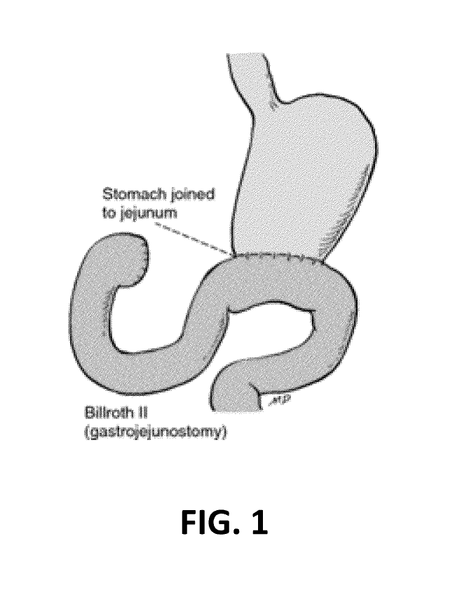

The term “gastrojejunostomy” refers to the surgical formation of a direct communication between the stomach and the jejunum, wherein the anastomosis (connection) is created. It is a procedure in which the duodenum is bypassed and the stomach is anastomosed to the jejunum. As such, gastrojejunostomy can refer to the surgical creation of an anastomosis between the stomach and jejunum, as shown in FIG. 1.

As used herein, “stoma” or “gastrojejunostomy stoma” refers to an outlet, pore or opening that is created in the gastric pouch and leads into the attached jejunum during a gastrojejunostomy. The term embraces the outlet itself, as well as the tissue walls surrounding the outlet.

As used herein, “anastomosis” refers to the connection of two structures. It includes connections between blood vessels or between other tubular structures such as loops of intestine. In circulatory anastomoses, many arteries naturally anastomose with each other, for example the inferior epigastric artery and superior epigastric artery. The circulatory anastomosis is further divided into arterial and venous anastomosis. Arterial anastomosis includes actual arterial anastomosis (e.g. palmar arch, plantar arch) and potential arterial anastomosis (e.g. coronary arteries and cortical branch of cerebral arteries). An example of surgical anastomosis occurs when a segment of intestine is resected and the two remaining ends are sewn or stapled together (anastomosed). The procedure is referred to as intestinal anastomosis.

A gastrojejunal anastomosis (GJA) refers to the combination of stoma, gastrojejunostomy and anastomosis created during a gastric bypass procedure.

A pathological anastomosis can result from trauma or disease and may involve veins, arteries, or intestines. These are usually referred to as fistulas. In the cases of veins or arteries, traumatic fistulas usually occur between artery and vein. Traumatic intestinal fistulas usually occur between two loops of intestine (entero-enteric fistula) or intestine and skin (enterocutaneous fistula). A portacaval anastomosis, by contrast, is a spontaneous or surgically created anastomosis between a vein of the portal circulation and a vein of the systemic circulation, which allows blood to bypass the liver in patients with portal hypertension.

As used herein, “bariatric surgery” or “bariatric surgical procedure” refers to a variety of procedures performed on people who are obese. Weight loss is achieved by reducing the size of the stomach with an implanted medical device (gastric banding) or through removal of a portion of the stomach (sleeve gastrectomy or biliopancreatic diversion with duodenal switch) or by resecting and re-routing the small intestines to a small stomach pouch (gastric bypass surgery). Bariatric surgery is the term encompassing all of the surgical treatments for morbid obesity, of which gastric bypass is only one class of such operations.

As used herein, “9-octadecanoic acid” refers to the 18 carbon chain fatty acid with a single degree of unsaturation (carbon-carbon double bond in the cis-configuration) between C-9 and C-10. The CAS Registry Number is 112-80-1. The compound is structurally illustrated below.

The 9-octadecanoic acid can exist as the free acid. Alternatively, the 9-octadecanoic acid can exist as a salt, formed from the combination of a suitable counterion and the 9-octadecanoic acid. In specific embodiments, the suitable counterion is ethanolamine.

The term “9-octadecanoic acid” includes relatively pure 9-octadecanoic acid, as well as those compositions that include 9-octadecanoic acid. For example, the term “9-octadecanoic acid” includes USP oleic acid NF, which is about 70-85 wt. % pure 9-octadecanoic acid, the remainder being other fatty acids. As used herein, “ethanolamine” refers to the compound 2-aminoethanol or monoethanolamine [CAS REG. NO. 141-43-5], which is an organic chemical compound that is both a primary amine and a primary alcohol (due to a hydroxyl group). Like other amines, monoethanolamine acts as a weak base. The compound is structurally illustrated below.

The ethanolamine can exist as the free base, or as an acid addition salt. When the ethanolamine exists as an acid addition salt, a counterion from the acid forms a salt with the ethanolamine. Suitable acids include, e.g., fatty acids, such as 9-octadecanoic acid.

Ethanolamine serves as a counter positive ion (cation) to solubilize oleic acid, which along with a small amount of solvent (e.g., benzyl alcohol) produces an acceptable clear solution for parenteral intra-tissue or intravenous injection.

The term “counter ion” refers to a charged atom (e.g., Br—) or group of atoms (e.g., an acetate or oleate anion), that forms a salt with a parent compound (e.g., ethanolamine) having an opposite charge.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The phrase “pharmaceutically acceptable salt” refers to pharmaceutically acceptable organic or inorganic salts of a compound (e.g., ethanolamine and/or 9-octadecanoic acid). The pharmaceutically acceptable salt is formed from a suitable counterion (e.g., anion or cation).

When the compound includes a base (e.g., ethanolamine), the desired pharmaceutically acceptable salt may be prepared by any suitable method available in the art, for example, treatment of the free base ethanolamine with an inorganic acid (e.g., hydrochloric acid, hydrobromic acid, sulfuric acid, nitric acid, methanesulfonic acid, phosphoric acid and the like), or with an organic acid (e.g., acetic acid, 9-octadecanoic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like). Additional suitable acids include, e.g., saturated and/or unsaturated fatty acids, such as trans- or cis-9-octadecanoic acid, oleic acid USP NF, stearic acid, tretradecyl, polydocanol, and morrhuate. The table below shows non-limiting examples of fatty acids suitable herein.

Examples of Unsaturated Fatty Acids

| Common | | |
|---|---|---|
| name | Chemical structure | Δ* |
| Mynristoleic acid | CH₃(CH₂)₃CH═CH(CH₂)₃COOH | cis-Δ⁸ |
| Palmitoleic acid | CH₃(CH₂)₃CH═CH(CH₂)₃COOH | cis-Δ⁸ |
| Sapienic acid | CH₃(CH₂)₃CH═CH(CH₂)₃COOH | cis-Δ⁸ |
| Oleic acid | CH₃(CH₂)₃CH═CH(CH₂)₃COOH | cis-Δ⁸ |
| Elaidic acid | CH₃(CH₂)₃CH═CH(CH₂)₃COOH | trans-Δ⁸ |
| Vaccenic | CH₃(CH₂)₃CH═CH(CH₂)₃COOH | trans-Δ¹² |
| acid | | |
| Linoleic acid | CH₃(CH₂)₄CH═CHCH₂CH═CH(CH₂)₇COOH | cis,cis-Δ⁸,Δ¹² |
| Linoelaidic acid | CH₃(CH₂)₄CH═CHCH₂CH═CH(CH₂)₇COOH | trans,trans-Δ⁸,Δ¹² |
| α-Linolenic acid | CH₃CH₂CH═CHCH₂CH═CHCH₂CH═CH(CH₂)₇COOH | cis,cis,cis-Δ⁸,Δ¹²,Δ¹⁶ |
| Arachidonic acid | CH₃(CH₂)₄CH═CHCH₂CH═CHCH₂CH═CHCH₂CH═CH(CH₃)₃COOH^{NIST}-n | cis,cis,cis,cis- |
| | | Δ⁴,Δ⁸,Δ¹²,Δ¹⁴ |
| Eicosapentaenoic | CH₃CH₂CH═CHCH₂CH═CHCH₃CH═CHCH₂CH═CHCH₂CH═CH(CH₂)₂COOH | cis,cis,cis,cis,cis- |
| acid | | Δ⁴,Δ⁸,Δ¹²,Δ¹⁴,Δ¹⁸ |
| Erucic acid | CH₂(CH₂)₇CH═CH(CH₃)₃COOH | cis-Δ¹³ |
| Docosahexaenoic | CH₃CH₃CH═CHCH₂CH═CHCH₂CH═CHCH₂CH═CHCH₂CH═CHCH₂CH═CH(CH₂)₂COOH | cis,cis,cis,cis,cis,cis- |
| acid | | Δ⁴,Δ⁸,Δ¹²,Δ¹⁴,Δ¹⁶,Δ¹⁸ |

When the compound includes an acid (e.g., 9-octadecanoic acid), the desired pharmaceutically acceptable salt may be prepared by any suitable method, for example, treatment of the free acid 9-octadecanoic acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. In specific embodiments, the counterion amine is ethanolamine.

For a review on pharmaceutically acceptable salts, see, e.g., Berge et al., J. Pharm. Sci. 1977, 66(1), 1-19, which is incorporated herein by reference.

The phrase “pharmaceutically acceptable” indicates that the substance or composition must be compatible chemically and/or toxicologically, with the other ingredients comprising a formulation, and/or the human being treated therewith.

The pharmaceutically acceptable salts of the compound ethanolamine can be synthesized by conventional chemical methods. Generally, such salts can be prepared by reacting the free base form of the parent compound with a stoichiometric amount of the appropriate acid, in water or in an organic solvent, or in a mixture of the two; generally, nonaqueous media like ether, ethyl acetate, ethanol, isopropanol, or acetonitrile are preferred. Lists of many suitable salts are found in Remington's Pharmaceutical Sciences, 17th ed., Mack Publishing Company, Easton, Pa., (1985), 1418, and the disclosure of which is incorporated herein by reference.

As used herein, “morrhuate” refers to saturated and unsaturated fatty acids of cod liver oil.

As used herein, “ethanolamine oleate” or “ethanolamine 9-octadecanoic acid” refers to the oleic acid salt of ethanolamine, as structurally shown below:

As used herein, “morrhuate ethanolamine” refers to the morrhuate salt of ethanolamine. In various embodiments, morrhuate ethanolamine can be employed in the methods described herein, instead of (or in addition to) the ethanolamine and 9-octadecanoic acid, or pharmaceutically acceptable salt thereof (e.g., ethanolamine oleate). For example, the morrhuate ethanolamine can be administered to the stomach of a human, in the various embodiments described herein, for the treatment of weight control.

As used herein, “inject” or “injection” refers to an infusion method of putting fluid into the body, usually with a hollow needle and a syringe, which is pierced through the skin to a sufficient depth for the material to be forced into the body.

As used herein, “endoscopic” or “endoscopy” refers to looking inside, and typically refers to looking inside the body for medical reasons using an endoscope, an instrument used to examine the interior of a hollow organ or cavity of the body. Unlike most other medical imaging devices, endoscopes can be inserted directly into the organ. Endoscopy can also refer to using a borescope in technical situations where direct line of-sight observation is not feasible.

As used herein, “sclerotherapy” refers to procedure used to treat blood vessels or blood vessel malformations (vascular malformations) and also those of the lymphatic system. A medicine (e.g., ethanolamine and 9-octadecanoic acid, or a pharmaceutically acceptable salt thereof, such as ethanolamine oleate)) is injected into the vessels, which induces inflammation in the wall of the vessel and results in thrombus formation and occlusion of the vessel. Injection of the “sclerosant” or sclerosing medicine directly into the tissue alongside the vessel causes, inflammation in the tissue, scarring and contraction of the tissue surrounding the injection site.

As used herein, “circumferentially injected” refers to an injection substantially along the circumference of an organ or tissue.

Hypodermic needles are available in a wide variety of outer diameters described by gauge numbers. Smaller gauge numbers indicate larger outer diameters. Inner diameter depends on both gauge and wall thickness. As such, as used herein, “gauge” refers to the outer diameter of a needle.

As used herein, “gastric bypass surgery” or “GBP” refers to a group of similar operations that first divides the stomach into a small upper pouch, or stoma, and a much larger lower “remnant” pouch. The stoma is connected to the small intestine via a sutured anastomosis. Surgeons have developed several different ways to reconnect the intestine, thus leading to several different GBP names. Any GBP leads to a marked reduction in the functional volume of the stomach, accompanied by an altered physiological and physical response to food that results in early postprandial satiety and malabsorption. The operation is prescribed to treat morbid obesity (defined as a body mass index greater than 40), type 2 diabetes, hypertension, sleep apnea, and other comorbid conditions linked to obesity. The resulting weight loss, typically dramatic, markedly reduces comorbidities. The long-term mortality rate of gastric bypass patients has been shown to be reduced by as much as 40%. As with all surgery, complications can occur. A study from 2005-2006 revealed that 15% of patients experience complications as a result of gastric bypass, and 0.5% of patients died within six months of surgery due to complications. A common form of gastric bypass surgery is the Roux-en-Y gastric bypass.

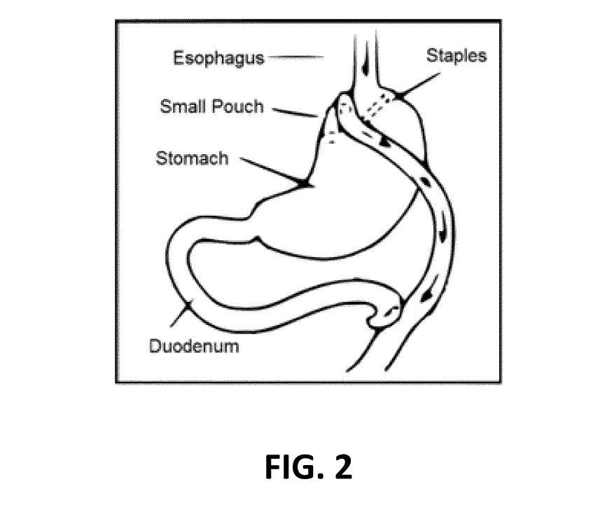

As used herein, “Roux-en Y gastric bypass” or “RYGB” surgery refers to a type of gastric bypass surgery in which a small stomach pouch is created with a stapler device, and connected to the small intestine thus bypassing the larger portion of the distal stomach, the first part of the small intestine called the duodenum, and the proximal jejunum. See FIG. 2. This bypassed segment is then reattached to the small intestine coming from the proximal gastric pouch in a Y-shaped configuration. During this surgical procedure, the stomach is divided into two compartments. The upper part of the stomach, called a pouch, receives the food that you eat. This new pouch is approximately 15 cubic centimeters (or the size of a golf ball). While the surgery reduces the capacity of the stomach by 90-95 percent, no part of the stomach is removed. Next, an outlet, called a stoma, is created in the upper small stomach pouch. This outlet is about 12 millimeters in diameter—about the size of an “M&M”. The food a person eats must be able to pass through this opening, which is why it must be chewed very well. Once through the opening, the food enters the loop of small intestine that has been brought up and joined to the new pouch and stoma. Food thus bypasses the lower part of the stomach and a very small part of the small intestine called the duodenum and proximal jejunum (which is why the surgical procedure is also known as the “gastric bypass”). Digestion and absorption occurs in the remainder of the intestine.

As used herein, “body mass index” or “BMI” refers to a heuristic proxy for human body fat based on an individual's weight and height. BMI does not actually measure the percentage of body fat. It was devised by the Belgian polymath Adolphe Quetelet during the course of developing “social physics.” Body mass index is defined as the individual's body mass divided by the square of his or her height. The formulae universally used in medicine produce a unit of measure of kg/m². BMI can also be determined using a BMI chart, which displays BMI as a function of weight (horizontal axis) and height (vertical axis) using contour lines for different values of BMI or colors for different BMI categories.

As used herein, “recurrent weight gain” refers to an increase in weight, e.g., following bariatric surgery. The increase can be, e.g., up to about 5%, up to about 10%, or up to about 20% of the human patient's weight initially lost after the bariatric surgery. The term refers to weight that was initially lost following bariatric surgery, but subsequently gained. This can occur over an extended period of time (e.g., up to a year, up to two years, up to five years, up to ten years, or up to twenty years). It is advantageous and desirable that human patients having previously experienced bariatric surgery, any recurrent weight gain will be less than about 20%, less than about 10%, less than about 5%, or less than about 1%.

As used herein, “weight stabilization” refers to a relatively little or no increase in weight, e.g., following bariatric surgery. This can occur over an extended period of time (e.g., up to a year, up to two years, up to five years, up to ten years, or up to twenty years). For example, if an increase in weight does occur, the increase will be no more than about 5%, no more than about 2.5%, or no more than about 1% of the human patient's weight. It is advantageous and desirable that human patients having previously experienced bariatric surgery, they will experience weight stabilization, such that any increase in the patient's weight will be no more than about 5%, no more than about 2.5%, or no more than about 1%, relative to the patient's weight.

As used herein, “weight loss” refers to a decrease in weight, e.g., following bariatric surgery. This can occur over an extended period of time (e.g., up to a year, up to two years, up to five years, up to ten years, or up to twenty years). The decrease can be, e.g., greater than about 1%, greater than about 5%, greater than about 10%, or greater than about 20% of the human patient's weight. It is advantageous and desirable that human patients having previously experienced bariatric surgery, they lose weight in an amount of greater than about 1%, greater than about 5%, greater than about 10%, or greater than about 20%, relative to the patient's weight.

As used herein, “dysfunctional eating behavior” refers to those eating behaviors or eating habits widely considered by the medical profession to be relatively unhealthy. This includes either excessive food intake, or the intake of foods lacking sufficient nutritional content, to the detriment of an individual's physical and/or mental health. Examples include binge eating disorder (BED), compulsive overeating, as well as a diet high in fat, sugar, and/or starch.

As used herein, “ghrelin” refers to a 28 amino acid hunger-stimulating peptide and hormone that is produced mainly by P/D1 cells lining the fundus of the human stomach and epsilon cells of the pancreas. Ghrelin together with obestatin is produced from cleavage of the ghrelin/obestatin prepropeptide (also known as the appetite-regulating hormone or growth hormone secretagogue or motilin-related peptide) which in turn is encoded by the GHRL gene.

As used herein, “perforation” refers to a small hole or aperture in a bodily membrane (e.g., gastrojejunostomy stoma).

As used herein, “ulceration” refers to discontinuity or break in the lining of an organ (e.g. an ulceration in the gastrojejunostomy stoma) that is the result of inflammation and can cause swelling, bleeding and scarring in the tissue that impedes the normal function of the organ.

As used herein, “stricture” refers to a tightening of a lumen or opening between two organs (e.g., the gastrojejunostomy stoma) that is the result of inflammation, swelling and eventual scarring in the tissue. Following, e.g., Roux-en-Y Gastric Bypass operation, scarring at the pouch's outlet can cause the outlet to narrow. This is called stomal stenosis, or stricture.

### Pharmaceutical Formulation

The parent compound or active ingredient (e.g., ethanolamine and 9-octadecanoic acid, or a pharmaceutically acceptable salt thereof (e.g., ethanolamine oleate)), can be formulated with conventional carriers and/or excipients, which will be selected in accord with ordinary practice. All formulations will optionally contain excipients such as those set forth in the Handbook of Pharmaceutical Excipients, 5th Ed.; Rowe, Sheskey, and Owen, Eds.; American Pharmacists Association; Pharmaceutical Press: Washington, D.C., 2006. The pH of the formulations ranges from about 3 to about 11, but is ordinarily about 7 to 10.

While it is possible for the active ingredient (e.g., ethanolamine oleate) to be administered alone, it may be preferable to present it as pharmaceutical formulation. The formulations include at least one active ingredient such as a long chain fatty acid soap, as above defined, together with one or more acceptable carriers and optionally other inactive ingredients. The carrier(s) will be “acceptable” in the sense of being compatible with the other ingredients of the formulation and physiologically innocuous to the recipient thereof or enhancing of the inflammatory effect of the long chain fatty acid.

The pharmaceutical compositions of the disclosed subject matter can be in the form of a sterile injectable preparation, such as a sterile injectable aqueous or oleaginous suspension. This suspension may be formulated according to the known art using those suitable dispersing or wetting agents and suspending agents. See, Techniques and formulations generally are found in Remington's Pharmaceutical Sciences, Mack Publishing Company, Easton, Pa., (1985). The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, solubility is greatly enhanced and the character of the solution made bacteriostatic or bacteriocidal by the addition of benzyl alcohol. Other such alcohols or solubilizing agents can also be readily utilized to infer similar properties as the benzyl alcohol.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies. It can be expected that the amount can include from about 0.0001 to about 100 mg/kg body weight, per dose. Specifically, the amount can include from about 0.01 to about 10 mg/kg body weight, per dose. More specifically, the amount can include from about 0.01 to about 5 mg/kg body weight, per dose. More specifically, the amount can include from about 0.05 to about 0.5 mg/kg body weight, per dose. For example, the dose for an adult human of approximately 70 kg body weight will range from 1 mg to 1000 mg, preferably between 5 mg and 500 mg, and may take the form of single or multiple doses.

Pharmaceutical kits useful in the presently disclosed subject matter, which include a therapeutically effective amount of a pharmaceutical composition that includes the parent compound of component (a) and one or more compounds of component (b), in one or more sterile containers, are also within the ambit of the presently disclosed subject matter. Sterilization of the container may be carried out using conventional sterilization methodology well known to those skilled in the art. Component (a) and component (b) may be in the same sterile container or in separate sterile containers. The sterile containers or materials may include separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit. The sterile containers included within the kit can include one or more syringes, one or more needles, and/or one or more vials.

### Utility

The compositions described herein are administered via injection to the stomach of a human. In specific embodiments, the human patient has previously undergone surgery (e.g., bariatric surgery) to promote weight loss. In such embodiments, the composition is administered via injection to post-bariatric surgery patients having dilated GJA. In alternative specific embodiments, the composition is administered via injection to a human patient currently undergoing surgery (e.g., bariatric surgery) to promote weight loss. In alternative specific embodiments, the composition is administered via injection to a human patient that has neither undergone, nor is currently undergoing, surgery (e.g., bariatric surgery) to promote weight loss.

In specific embodiments, the composition described herein includes ethanolamine (or a pharmaceutically acceptable salt thereof) and 9-octadecanoic acid (or a pharmaceutically acceptable salt thereof). In additional specific embodiments, the composition described herein includes a saturated or unsaturated fatty acid salt of ethanolamine. In additional specific embodiments, the composition described herein includes ethanolamine (or a pharmaceutically acceptable salt thereof) and 9-octadecanoic acid (or a pharmaceutically acceptable salt thereof), wherein each of the pharmaceutically acceptable salts are selected, such that the composition includes ethanolamine oleate. In additional specific embodiments, the composition described herein includes a morrhuate salt of ethanolamine. Unlike the sodium salt published with sodium morrhuate, ethanolamine serves to enhance the irritation, inflammation and sclerosing action of ethanolamine morrhuate used so it is more effective.

The administration, via injection, of ethanolamine and 9-octadecanoic acid, or a pharmaceutically acceptable salt thereof (e.g., ethanolamine oleate) will result in at least one of: (i) a decrease in the diameter of the GJA, (ii) a reduction of the stoma size, (iii) reduction of the stoma expansion, (iv) a reduction of the gastric pouch size, (v) the weight of the patient becoming stabilized, (vi) an increase in weight loss, (vii) a decrease in weight gain, (viii) decrease in recurrent weight gain, (ix) prevention in recurrent weight gain, (x) prevention of diabetes, (xi) decrease in caloric intake, (xii), decrease in food intake, (xiii) decrease in meal size consumed, (xiv) decrease in the number of meals consumed, (xv) decrease in appetite, (xvi) inhibition in the action of a hunger-stimulating hormone, (xvii) inhibition in the action of an appetite-regulating hormone, (xviii) inhibition in the action of ghrelin, and (xix) lowering the level of ghrelin. In specific embodiments, any one or more of the above is achieved with little or no ulceration.

### ENUMERATED EMBODIMENTS

Enumerated embodiments [1]-[35] provided below are for illustration purposes only and do not otherwise limit the scope of the invention, as defined by the claims. The enumerated embodiments [1]-[35] described below encompass all combinations and sub-combinations, whether or not expressly described as such.
[1.] A method including administering via injection ethanolamine and 9-octadecanoic acid, or a pharmaceutically acceptable thereof (e.g., ethanolamine oleate), to the stomach of a human.
[2.] The method of the above embodiment, wherein the ethanolamine and 9-octadecanoic acid, or a pharmaceutically acceptable thereof (e.g., ethanolamine oleate) is administered to at least one of the gastrojejunostomy stoma, cardia, fundus and intestine of the human patient.
[3.] The method of any one of the above embodiments, which is a method of at least one of:
   decreasing the diameter of the GJA in a human patient;
   reducing stoma size in a human patient;
   reducing the gastric pouch size in a human patient;
   reducing stoma expansion in a human patient;
   hardening the stomach in a human patient;
   stabilizing the weight of a human patient;
   increasing weight loss in a human patient;
   decreasing weight gain in a human patient;
   decreasing recurrent weight gain in a human patient;
   preventing recurrent weight gain in a human patient;
   preventing diabetes in a human patient;
   decreasing the caloric intake in a human patient;
   decreasing food intake in a human patient;
   decreasing a meal size consumed by a human patient;
   decreasing the number of meals consumed by a human patient;
   decreasing the appetite of a human patient;
   inhibiting the action of a hunger-stimulating hormone in a human patient;
   inhibiting the action of an appetite-regulating hormone in a human patient;
   inhibiting the action of ghrelin in a human patient; and
   lowering the level of ghrelin in a human patient.
[4.] The method of any one of the above embodiments, wherein prior to the administration, the human previously experienced at least one of:
   bariatric surgery;
   bariatric surgery, with recurrent weight gain or inadequate weight loss;
   gastric bypass surgery;
   gastric bypass surgery, with recurrent weight gain or inadequate weight loss;
   Roux-en Y gastric bypass (RYGB) surgery; and
   Roux-en Y gastric bypass (RYGB) surgery, with recurrent weight gain or inadequate weight loss.
[5.] The method of embodiment [4], wherein the surgery occurred up to about 10 years prior to the administration of the ethanolamine and 9-octadecanoic acid, or a pharmaceutically acceptable thereof (e.g., ethanolamine oleate).
[6.] The method of any one of the above embodiments, wherein prior to the administration of the ethanolamine and 9-octadecanoic acid, or a pharmaceutically acceptable thereof (e.g., ethanolamine oleate), the human patient exhibited dysfunctional eating behavior.
[7.] The method of any one of the above embodiments, wherein after the administration of the ethanolamine and 9-octadecanoic acid, or a pharmaceutically acceptable thereof (e.g., ethanolamine oleate), the human patient experiences minimal or no serious complications selected from perforation, ulceration, bleeding, stricture, and combinations thereof.
[8.] The method of any one of the above embodiments, wherein ethanolamine and 9-octadecanoic acid, or a pharmaceutically acceptable thereof (e.g., ethanolamine oleate) is administered to the gastrojejunostomy stoma of the human.
[9.] The method of any one of the above embodiments, wherein ethanolamine and 9-octadecanoic acid, or a pharmaceutically acceptable thereof (e.g., ethanolamine oleate) is administered multiple times to the gastrojejunostomy stoma of the human.
[10.] The method of any one of the above embodiments, wherein the ethanolamine and 9-octadecanoic acid, or a pharmaceutically acceptable thereof (e.g., ethanolamine oleate) is admixed with a pharmaceutically acceptable carrier or excipient.
[11.] The method of any one of the above embodiments, wherein the ethanolamine and 9-octadecanoic acid, or a pharmaceutically acceptable thereof (e.g., ethanolamine oleate) is injected into the gastrojejunostomy stoma of the human.
[12.] The method of any one of the above embodiments, wherein the ethanolamine and 9-octadecanoic acid, or a pharmaceutically acceptable thereof (e.g., ethanolamine oleate) is administered to the human via an endoscopic injection, administered through a catheter and a needle emanating from the biopsy channel of the endoscope.
[13.] The method of any one of the above embodiments, wherein the ethanolamine and 9-octadecanoic acid, or a pharmaceutically acceptable thereof (e.g., ethanolamine oleate) is administered to the human via a sclerotherapy injection.
[14.] The method of any one of the above embodiments, wherein the ethanolamine and 9-octadecanoic acid, or a pharmaceutically acceptable thereof (e.g., ethanolamine oleate) is administered to the human via an endoscopic sclerotherapy injection.
[15.] The method of any one of the above embodiments, wherein a total of up to about 30 mL of ethanolamine and 9-octadecanoic acid, or a pharmaceutically acceptable thereof (e.g., ethanolamine oleate) is injected into the gastrojejunostomy stoma of the human.
[16.] The method of any one of the above embodiments, wherein the ethanolamine and 9-octadecanoic acid, or a pharmaceutically acceptable thereof (e.g., ethanolamine oleate) is circumferentially injected along the gastrojejunostomy stoma of the human.
[17.] The method of any one of the above embodiments, wherein the ethanolamine and 9-octadecanoic acid, or a pharmaceutically acceptable thereof (e.g., ethanolamine oleate) is circumferentially injected along the gastrojejunostomy stoma of a human, in about 2-4 ml per injection, in each of the four quadrants around the stoma.
[18.] The method of any one of the above embodiments, wherein the ethanolamine and 9-octadecanoic acid, or a pharmaceutically acceptable thereof (e.g., ethanolamine oleate) is circumferentially injected along the gastrojejunostomy stoma of a human, in about 4-18 injections.
[19.] The method of any one of the above embodiments, wherein the ethanolamine and 9-octadecanoic acid, or a pharmaceutically acceptable thereof (e.g., ethanolamine oleate) is injected into the gastrojejunostomy of a human, employing a needle having a gauge of about 23 to about 25 guage.
[20.] The method of any one of the above embodiments, wherein prior to administration of the ethanolamine and 9-octadecanoic acid, or a pharmaceutically acceptable thereof (e.g., ethanolamine oleate), the gastrojejunostomy anastamosis is dilated, having a diameter of greater than about 12 mm.
[21.] The method of any one of the above embodiments, wherein a therapeutically effective amount of ethanolamine and 9-octadecanoic acid, or a pharmaceutically acceptable thereof (e.g., ethanolamine oleate) is administered to decrease the diameter of the gastrojejunostomy anastamosis to no less than about 10 mm.
[22.] The method of any one of the above embodiments, wherein the human is obese, having a body mass index (BMI) of at least about 30.
[23.] The method of any one of the above embodiments, further including, prior to the administration of the ethanolamine and 9-octadecanoic acid, or a pharmaceutically acceptable thereof (e.g., ethanolamine oleate) performing an endoscopy.
[24.] The method of embodiment [23], which is carried out two or more times.
[25.] The method of embodiment [23], wherein the endoscopy is performed to measure the size of the diameter of the gastrojejunostomy anastamosis.
[26.] The method of any one of the above embodiments, wherein after the administration of the ethanolamine and 9-octadecanoic acid, or a pharmaceutically acceptable thereof (e.g., ethanolamine oleate), the human experiences weight stabilization or weight loss.
[27.] The method of any one of the above embodiments, wherein after the administration of the ethanolamine and 9-octadecanoic acid, or a pharmaceutically acceptable thereof (e.g., ethanolamine oleate), the human experiences weight stabilization or weight loss during the following 12 months.
[28.] The method of any one of the above embodiments, wherein a therapeutically effective amount of ethanolamine and 9-octadecanoic acid, or a pharmaceutically acceptable thereof (e.g., ethanolamine oleate) is administered to decrease recurrent weight gain, prevent recurrent weight gain, or provide for weight loss.
[29.] The method of any one of the above embodiments, wherein a therapeutically effective amount of ethanolamine and 9-octadecanoic acid, or a pharmaceutically acceptable thereof (e.g., ethanolamine oleate) is administered to decrease recurrent weight gain, prevent recurrent weight gain, or provide for weight loss, during the following 12 months.
[30.] The method of any one of the above embodiments, wherein after the administration of the ethanolamine and 9-octadecanoic acid, or a pharmaceutically acceptable thereof (e.g., ethanolamine oleate), the human patient experiences a weight loss, at a rate of at least about 1.0 kg/month.
[31.] The method of any one of the above embodiments, wherein after the administration of the ethanolamine and 9-octadecanoic acid, or a pharmaceutically acceptable thereof (e.g., ethanolamine oleate), the human patient experiences a weight loss, at a rate of up to about 0.75 kg/month.
[32.] The method of any one of the above embodiments, resulting in a relatively low systemic absorption of the ethanolamine and 9-octadecanoic acid, or a pharmaceutically acceptable thereof (e.g., ethanolamine oleate), such that any blood level increase resulting from the administration thereof is less than about 5 wt. %.
[33.] The method of any one of the above embodiments, resulting in a relatively low systemic absorption of the ethanolamine and 9-octadecanoic acid, or a pharmaceutically acceptable thereof (e.g., ethanolamine oleate), such that any blood level increase resulting from the administration thereof is less than about 1 wt. %.

[34.] The method of any one of the above embodiments, wherein the injectable solution does not cause clinically significant adverse safety effects, such that the injection is considered to be safe for administration to humans.
[35.] The method of any one of the above embodiments, wherein the injectable solution does not cause clinically significant adverse safety effects, such that the injection is approved by the US FDA to be safe and effective for administration to humans.

### EXAMPLES

### Example 1

### Pig Weight Gain over 5 Day Period

Approximately 30 kg three month old pigs are weighed and anesthetized. An endoscope is inserted, the tissue washed of stomach debris, then used to inject the region of the stomach with up to 16 injections of 2-4 mL each to induce inflammation, sclerosis, and scarring of the tissue. Weight gain measurements or repeat endoscopy injections can be conducted serially over several additional months, or annually, to achieve optimal results of this therapy. We also can examine the best regions, regimens, dose schedule, dose volume, ghrelin levels, along with other plasma parameters including plasma levels of ethanolamine and oleate. In addition the safety can be assessed to ensure no stomach ulceration, or other adverse clinical effects occur or detract from the primary goal of weight control. In this example below we have injected ethanolamine oleate into the fundus and cardia region, or into the fundus region, of normal pigs to assess the impact of the ethanolamine oleate formulation.

| | | Initial | Weight at | Weight | Weight | Injection |
|---|---|---|---|---|---|---|
| Pig | Injection and | Weight | 1 week | Gain | Gain | Site in |
| Tag # | Volume (ml) | (kg) | (kg) | (kg) | (%) | Stomach |
| 37725 | 32 ml | 29.3 kg | 31 kg | 1.7 kg | 5.8% | fundus |
| | ethanolamine | | | | | andcardia |
| | oleate | | | | | |
| 37724 | 32 ml saline | 30 kg | 32.5 kg | 2.5 kg | 8.3% | fundus |
| | | | | | | andcardia |
| 37722 | 28.5 ml | 30.5 kg | 31 kg | 0.5 kg | 1.6% | fundus |
| | ethanolamine | | | | | |
| | oleate | | | | | |
| 37723 | 28 ml saline | 31.9 kg | 33 kg | 1.1 kg | 3.4% | fundus |

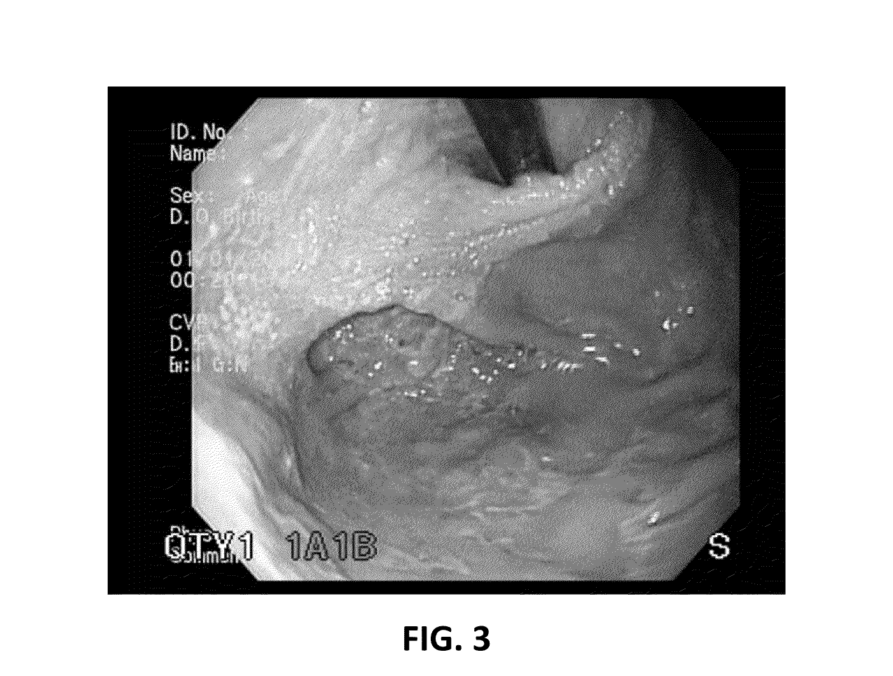

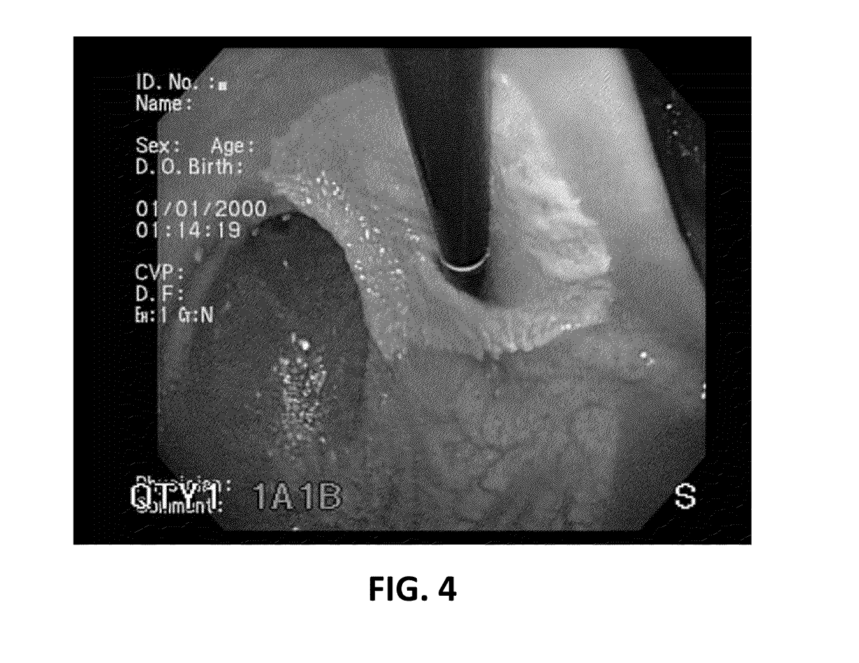

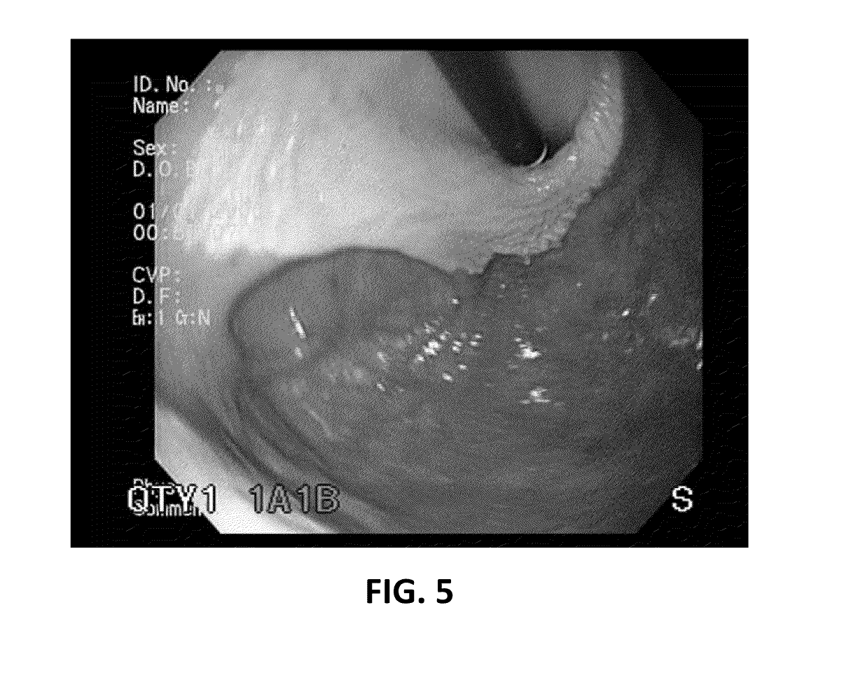

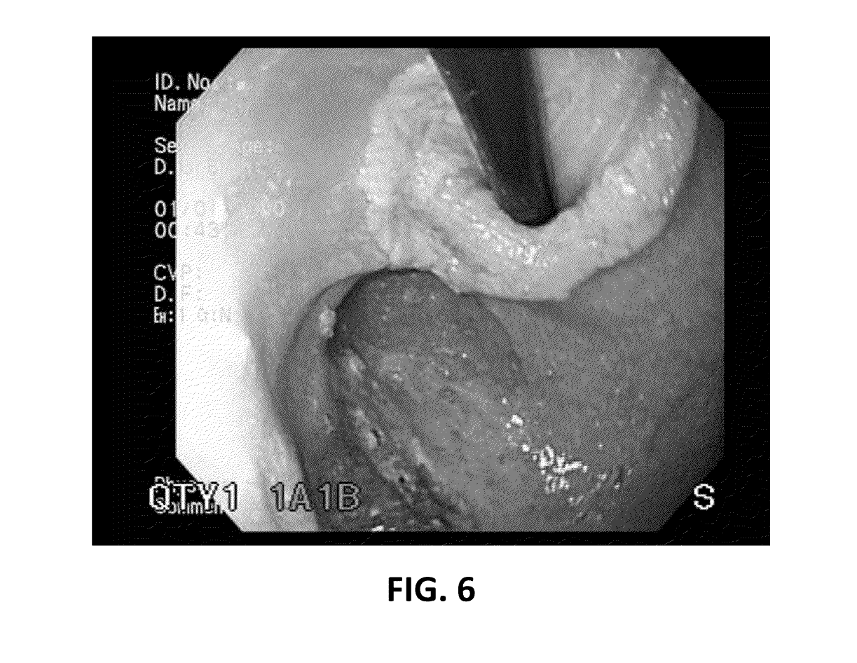

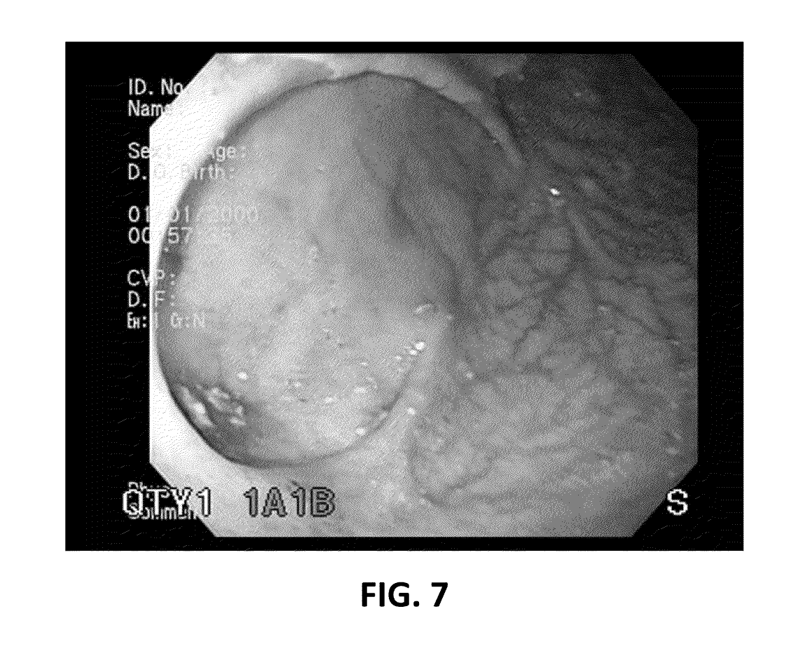

Photos of the injected regions at week one are shown as screen shots taken from endoscopy videos in FIGS. 3-7. A closer look at the mucosa of the fundus and cardia injected pig is also included. There is some nodularity seen on endoscopic examination in the ethamoline oleate treated pigs. However no obvious ulceration, necrosis or other lesions is evident. Insufflation of the stomach reveals decreased distensibility of the proximal stomach in the pigs treated with ethamoline oleate compared to the saline control. No differences are noted in the tissue for the two pigs injected in the cardia region.

TABLE 2
Plasma levels of ethanolamine and oleate immediately after injection
(time 0, and 10, 30 and 60 minutes later).

| | Injection | Time 0 end | | Time = | Time = | |
|---|---|---|---|---|---|---|
| | and | of injection | Time = | 30 | 60 | Injection |
| Pig | Volume | Micro- | 10 min | minutes | minutes | Site in |
| Tag # | (ml) | grams/mL | μg/mL | μg/mL | μg/mL | Stomach |
| 37725 | 32 ml | 2.55 ETA | 4.56 | 3.22 | 2.44 | cardia |
| | ethanol- | 8.79 Oleate | ETA | ETA | ETA | |
| | amine | | 7.43 | 6.27 | 5.48 | |
| | oleate | | Oleate | Oleate | Oleate | |
| 37724 | 32 ml | 0.425 ETA | 0.429 | 0.481 | 0.370 | cardia |
| | saline | 8.83 Oleate | ETA | ETA | ETA | |
| | | | 5.98 | 5.44 | 5.23 | |
| | | | Oleate | Oleate | Oleate | |
| 37722 | 28.5 ml | 2.08 ETA | 2.79 | 1.42 | 1.71 | fundus |
| | ethanol- | 15.6 Oleate | ETA | ETA | ETA | |
| | amine | | 11.9 | 7.80 | 7.05 | |
| | oleate | | Oleate | Oleate | Oleate | |
| 37723 | 28 ml | 0.388 ETA | 0.552 | 0.324 | 0.200 | fundus |
| | saline | 7.76 Oleate | ETA | ETA | ETA | |
| | | | 7.84 | 5.94 | 7.34 | |
| | | | Oleate | Oleate | Oleate | |

ETA = ethanolamine;
Oleate = 9-octadecanoic acid

To assess the safety of this procedure a plasma assay was validated to measure the increase in basal levels of endogenous ethanolamine or 9-octadecanoic acid. It was determined in this series that the basal or normal level in pigs treated with saline alone was on the order of 300-500 nanograms per milliliter of plasma for ethanolamine and 5-9 micrograms per milliliter (μg/mL) of pig plasma for 9-octadecanoic acid. The safety of this procedure, particularly with ethanolamine oleate is validated by 1) the presence of endogenous chemicals in plasma identical to the active ingredients, ethanolamine and 9-octadecanoic acid, and 2) that the levels after injection of a more than double the human dose on a weight basis (30 kg pigs versus human >60 kg body weight) are increased only slightly from basal levels to 1.42-4.56 μg/mL for ethanolamine (3-15 fold), and to 8-16 μg/mL for 9-octadecanoic acid which is no more than double the basal level. Further the pigs are reported to be clinically identical in their eating and their behavior following injection with either saline or ethanolamine oleate with no obvious signs or symptoms of any adverse events. The treating physician endoscopically examined each pig in this study at one week post injection, and noted no obvious abnormalities such as ulceration or injury at any injection sites in any pigs, but did notice less resiliency in the stomach wall for the ethanolamine oleate treated pigs implying efficacy consistent with the reduced weight gain for the drug treated pigs. Further, we expect and anticipate an impact on the Ghrelin levels from ethanolamine oleate treatment as well as continued differentiation of weight gain between treated and saline control pigs as this study progresses.

All publications, patents, and patent applications are incorporated herein by reference. While in the foregoing specification this invention has been described in relation to certain specific embodiments thereof, and many details have been set forth for purposes of illustration, it will be apparent to those skilled in the art that the invention can include additional embodiments, and that certain of the details described herein may be varied considerably without departing from the basic principles of the invention. The present invention can be illustrated by the following non-limiting example.

What is claimed is:

1. A method comprising administering via injection, ethanolamine oleate, to the stomach of a human.

2. The method of claim 1, wherein the ethanolamine oleate is administered to at least one of the gastrojejunostomy stoma, cardia, fundus and intestine of the human patient.

3. The method of claim 1, which is a method of at least one of:
decreasing the diameter of the GJA in a human patient;
reducing stoma size in a human patient;
reducing the gastric pouch size in a human patient;
reducing stoma expansion in a human patient;
hardening the stomach in a human patient;
stabilizing the weight of a human patient;
increasing weight loss in a human patient;
decreasing weight gain in a human patient;
decreasing recurrent weight gain in a human patient;
preventing recurrent weight gain in a human patient;
preventing diabetes in a human patient;
decreasing the caloric intake in a human patient;
decreasing food intake in a human patient;
decreasing a meal size consumed by a human patient;
decreasing the number of meals consumed by a human patient;
decreasing the appetite of a human patient;
inhibiting the action of a hunger-stimulating hormone in a human patient;
inhibiting the action of an appetite-regulating hormone in a human patient;
inhibiting the action of ghrelin in a human patient; and
lowering the level of ghrelin in a human patient.

4. The method of claim 1, wherein prior to the administration, the human previously experienced at least one of:
bariatric surgery;
bariatric surgery, with recurrent weight gain or inadequate weight loss;
gastric bypass surgery;
gastric bypass surgery, with recurrent weight gain or inadequate weight loss;
Roux-en Y gastric bypass (RYGB) surgery; and
Roux-en Y gastric bypass (RYGB) surgery, with recurrent weight gain or inadequate weight loss.

5. The method of claim 1, wherein prior to the administration of the ethanolamine oleate, the human patient exhibited dysfunctional eating behavior.

6. The method of claim 1, wherein after the administration of the ethanolamine oleate, the human patient experiences minimal or no serious complications selected from perforation, ulceration, bleeding, stricture, and combinations thereof.

7. The method of claim 1, wherein ethanolamine oleate is administered to the gastrojejunostomy stoma of the human.

8. The method of claim 1, wherein the ethanolamine oleate is admixed with a pharmaceutically acceptable carrier or excipient.

9. The method of claim 1, wherein the ethanolamine oleate is injected into the gastrojejunostomy stoma of the human.

10. The method of claim 1, wherein the ethanolamine oleate is administered to the human via an endoscopic injection, administered through a catheter and a needle emanating from the biopsy channel of the endoscope.

11. The method of claim 1, wherein the ethanolamine oleate is administered to the human via a sclerotherapy injection.

12. The method of claim 1, wherein the ethanolamine oleate is administered to the human via an endoscopic sclerotherapy injection.

13. The method of claim 1, wherein a total of up to about 30 mL of ethanolamine oleate is injected into the gastrojejunostomy stoma of the human.

14. The method of claim 1, wherein the ethanolamine oleate is circumferentially injected along the gastrojejunostomy stoma of the human.

15. The method of claim 1, wherein the ethanolamine oleate is circumferentially injected along the gastrojejunostomy stoma of a human, in about 2-4 ml per injection, in each of the four quadrants around the stoma.

16. The method of claim 1, wherein the ethanolamine oleate is circumferentially injected along the gastrojejunostomy stoma of a human, in about 4-18 injections.

17. The method of claim 1, wherein the ethanolamine oleate is injected into the gastrojejunostomy of a human, employing a needle having a gauge of about 23 to about 25 gauge.

18. The method of claim 1, wherein prior to administration of the ethanolamine oleate the gastrojejunostomy anastamosis is dilated, having a diameter of greater than about 12 mm.

19. The method of claim 1, wherein a therapeutically effective amount of ethanolamine oleate is administered to decrease the diameter of the gastrojejunostomy anastamosis to no less than about 10 mm.

20. The method of claim 1, wherein the human is obese, having a body mass index (BMI) of at least about 30.

21. The method of claim 1, further comprising, prior to the administration of the ethanolamine oleate performing an endoscopy.

22. The method of claim 21, wherein the endoscopy is performed to measure the size of the diameter of the gastrojejunostomy anastamosis.

23. The method of claim 1, wherein after the administration of the ethanolamine oleate the human experiences weight stabilization or weight loss.

24. The method of claim 1, wherein after the administration of the ethanolamine oleate the human experiences weight stabilization or weight loss during the following 12 months.

25. The method of claim 1, wherein a therapeutically effective amount of ethanolamine oleate is administered to decrease recurrent weight gain, prevent recurrent weight gain, or provide for weight loss.

26. The method of claim 1, wherein a therapeutically effective amount of ethanolamine oleate is administered to decrease recurrent weight gain, prevent recurrent weight gain, or provide for weight loss, during the following 12 months.

27. The method of claim 1, wherein after the administration of the ethanolamine oleate the human patient experiences a weight loss, at a rate of at least about 1.0 kg/month.

28. The method of claim 1, resulting in a relatively low systemic absorption of the ethanolamine oleate such that any blood level increase resulting from the administration thereof is less than about 1 wt. %.

29. The method of claim 1, wherein the injectable solution does not cause clinically significant adverse safety effects, such that the injection is considered to be safe for administration to humans.
